(19) United States
(12) Patent Application Publication
(10) Pub. No.: US 2025/0067719 A1
(43) Pub. Date: Feb. 27, 2025

(54) Gas Chromatography for Liquid Phase Light Hydrocarbon Detection

(71) Applicant: Halliburton Energy Services, Inc., Houston, TX (US)

(72) Inventor: Mathew Dennis Rowe, Houston, TX (US)

(21) Appl. No.: 18/237,298

(22) Filed: Aug. 23, 2023

(51) Int. Cl. G01N 33/28; B01D 29/52; B01D 53/02; E21B 21/06; E21B 49/08; G01N 30/14; G01N 30/30; G01N 30/32

(52) U.S. Cl. CPC G01N 33/2823; B01D 29/52; B01D 53/025; E21B 21/067; E21B 49/084; G01N 30/14; G01N 30/30; G01N 30/32; G01N 2030/025; G01N 2030/326

(57) ABSTRACT

A method for analyzing a drilling fluid used in a drilling operation within a subterranean formation by flowing the drilling fluid through a fluid conduit coupled to a drilling assembly; continuously flowing a drilling fluid sample of the drilling fluid from the fluid conduit, wherein the drilling fluid sample comprises a liquid; sending the drilling fluid sample to a bypass, or determining a chemical composition of the drilling fluid using gas chromatography (GC) of the drilling fluid sample, wherein determining the chemical composition of the drilling fluid using GC comprises: destructing the drilling fluid sample to provide a gaseous drilling fluid sample, wherein destructing the drilling fluid sample comprises converting one or more components (e.g., light hydrocarbons) of the drilling fluid sample to a gas; and introducing the gaseous drilling fluid sample to a GC system comprising a GC column to determine the chemical composition of the drilling fluid.

### TECHNICAL FIELD

The present disclosure relates generally to well drilling operations and, more particularly, to drilling fluid analysis using gas chromatography (GC).

### BACKGROUND

Wellbores are drilled from the earth's surface into a subterranean formation for the recovery of natural resources such as oil and gas. A wellbore is drilled by rotating a drill bit extending from the surface into the wellbore via a string of drill pipe. During drilling operations, a drilling fluid (also referred to drilling mud) is circulated down the interior of the drill pipe, out the drill bit, and back up the annulus between the drill pipe and wellbore wall, which aids in lubricating the drill bit and removing drill cuttings from the wellbore. For example, drilling fluids can be water based fluids (e.g., water based mud). Upon return to the surface, the drilling fluid can be analyzed to determine characteristics (e.g., a compositional parameter) thereof, which can be utilized to determine characteristics of the subterranean formation. The drilling fluid analysis may affect the accuracy and reliability of the analysis data and, therefore, the accuracy of the formation characteristics determined using the analysis data.

### BRIEF SUMMARY OF THE DRAWINGS

For a more complete understanding of this disclosure, reference is now made to the following brief description, taken in connection with the accompanying drawings and detailed description, wherein like reference numerals represent like parts.

FIG. 1 is a diagram of an example drilling system, according to aspects of the present disclosure;

FIG. 2 is a block diagram of an example information handling system, according to aspects of the present disclosure;

FIG. 3 is a block diagram of an example fluid analyzer, according to aspects of the present disclosure;

FIG. 4 is a block diagram of another example fluid analyzer, according to aspects of the present disclosure;

FIG. 5 is a schematic of an example sample preparation apparatus, according to aspects of the present disclosure;

FIG. 6 is a diagram of an example offshore drilling system, according to aspects of the present disclosure; and

FIG. 7 is a diagram of an example offshore drilling system, according to aspects of the present disclosure.

While embodiments of this disclosure are depicted and described and are defined by reference to example embodiments of the disclosure, such references do not imply a limitation on the disclosure, and no such limitation is to be inferred. The subject matter disclosed is capable of considerable modification, alteration, and equivalents in form and function, as will occur to those skilled in the pertinent art and having the benefit of this disclosure. The depicted and described embodiments of this disclosure are examples only, and not exhaustive of the scope of the disclosure.

### DETAILED DESCRIPTION

As noted above, during wellbore drilling operations, a drilling fluid (e.g., a water based mud or WBM) can be circulated (e.g., pumped) from a drilling rig located at the earth's surface downhole through a string of drill pipe supported by the drilling rig. The drilling fluid exits the drill pipe via a drill bit located at the end of the drill pipe and flows back uphole to the surface via an annular space located between the drill pipe and the wellbore wall. Upon return to the surface, the circulated, recovered drilling fluid can be further analyzed (also referred to as formation fluid characterization), which can be used to aid in assessing the subterranean formation and controlling (e.g., steering) drilling operations to improve oil and gas recovery and maximize asset value.

Conventionally, the determination of hydrocarbons in a formation has been limited in the gas phase due to extraction issues regarding extraction of the gas and liquid hydrocarbons from the recovered drilling fluid. By way of example, when extracting hydrocarbons from drilling fluid, methane may come out at 99% efficiency, but the extraction efficiency can drop dramatically with increasing hydrocarbon weight. For example, pentane may only extract at a 20% efficiency. This “extraction bias” can lead to a need to correct for extraction efficiency through different techniques that add cost and time to the process.

Herein disclosed are a system and method for analyzing, in gas phase, drilling fluid used in a drilling operation within a subterranean formation, for example, to determine a light hydrocarbon content thereof. The disclosed apparatus for analyzing a drilling fluid (also referred to herein as an, “analyzer”), system and method comprise placing a GC column of a GC system in fluid communication with a sample flow of the drilling fluid. The drilling fluid from which the sample flow is derived can be flowing through a fluid conduit coupled to a drilling assembly. A chemical composition of the drilling fluid can be determined using the GC system. Additionally, a formation characteristic can subsequently be determined using the determined chemical composition.

Via the analyzer, system and method of this disclosure, a pump can be connected to a drilling fluid flowline (e.g., a line or “fluid conduit” containing drilling fluid flowing into or out of the wellbore) to continuously extract a portion of the drilling fluid (e.g., from which a “drilling fluid sample” is obtained) from the fluid conduit. The extracted drilling fluid can flow through a drilling fluid sample preparation system (also referred to herein as a “sampling system”). The drilling fluid sample preparation system can comprise filters (e.g., a gross filter and a regenerative fine filter, as described further hereinbelow) to remove solids from the portion of the drilling fluid continuously extracted from the fluid conduit, to provide the drilling fluid sample. The filters can be regenerative and/or automatically replaced as needed to provide sufficient solids removal from the continuously extracted flow of the portion of the drilling fluid. The drilling fluid sample obtained from the sampling system can be automatically loaded in a flash column of a GC system or sent to bypass. When analysis is effected, and the drilling fluid sample is sent to the GC system, the drilling fluid sample is flashed to extract light hydrocarbon species as a gas from the drilling fluid sample comprising liquid, thus providing a gaseous drilling fluid sample. The flash column can be automatically replaced and/or regenerated. The gaseous drilling fluid sample comprising the flashed hydrocarbons are introduced to one or more gas chromatography (GC) column(s), for example using a carrier gas. The hydrocarbons can then be detected by one or more detectors, such as a flame ionization detector (FID), a thermal conductivity detector (TCD), a photo ionization detector (PID), another detector, or a combination thereof. The analysis can further include other techniques in addition to the GC. The sample results from the analyzer (comprising the GC results) can be tagged to a depth and transmitted to an information handling system (e.g., a computer). The real time (RT) or depth-based data from the analyzer can be utilized by the information handling system to determine a drilling efficiency or a formational fluid type (i.e., fluids, such as light hydrocarbons, in the subterranean formation).

For purposes of this disclosure, an information handling system may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, an information handling system may be a personal computer, a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communication with external devices as well as various input and output (I/O) devices, such as a keyboard, a mouse, and a video display. The information handling system may also include one or more buses operable to transmit communications between the various hardware components. It may also include one or more interface units capable of transmitting one or more signals to a controller, actuator, or like device.

For the purposes of this disclosure, computer-readable media may include any instrumentality or aggregation of instrumentalities that may retain data and/or instructions for a period of time. Computer-readable media may include, for example, without limitation, storage media such as a direct access storage device (e.g., a hard disk drive or floppy disk drive), a sequential access storage device (e.g., a tape disk drive), compact disk, CD-ROM, DVD, RAM, ROM, electrically erasable programmable read-only memory (EEPROM), and/or flash memory; as well as communications media such wires, optical fibers, microwaves, radio waves, and other electromagnetic and/or optical carriers; and/or any combination of the foregoing.

Illustrative embodiments of the present disclosure are described in detail herein. In the interest of clarity, not all features of an actual implementation may be described in this specification. It will of course be appreciated that in the development of any such actual embodiment, numerous implementation-specific decisions must be made to achieve the specific implementation goals, which will vary from one implementation to another. Moreover, it will be appreciated that such a development effort might be complex and time-consuming, but would nevertheless be a routine undertaking for those of ordinary skill in the art having the benefit of the present disclosure.

To facilitate a better understanding of the present disclosure, the following examples of certain embodiments are given. In no way should the following examples be read to limit, or define, the scope of the disclosure. Embodiments of the present disclosure may be applicable to drilling operations that include, but are not limited to, target (such as an adjacent well) following, target intersecting, target locating, well twinning such as in SAGD (steam assist gravity drainage) well structures, drilling relief wells for blowout wells, river crossings, construction tunneling, as well as horizontal, vertical, deviated, multilateral, u-tube connection, intersection, bypass (drill around a mid-depth stuck fish and back into the well below), or otherwise nonlinear wellbores in any type of subterranean formation. Embodiments may be applicable to injection wells, stimulation wells, and production wells, including natural resource production wells such as hydrogen sulfide, hydrocarbons or geothermal wells; as well as borehole construction for river crossing tunneling and other such tunneling boreholes for near surface construction purposes or borehole u-tube pipelines used for the transportation of fluids such as hydrocarbons. Embodiments described below with respect to one implementation are not intended to be limiting.

Modern petroleum drilling and production operations demand information relating to parameters and conditions downhole. Several methods exist for downhole information collection, including logging-while-drilling (“LWD”) and measurement-while-drilling (“MWD”). In LWD, data is typically collected during the drilling process, thereby avoiding any need to remove the drilling assembly to insert a wireline logging tool. LWD consequently allows the driller to make accurate real-time modifications or corrections to optimize performance while minimizing downtime. MWD is the term for measuring conditions downhole concerning the movement and location of the drilling assembly while the drilling continues. LWD concentrates more on formation parameter measurement. While distinctions between MWD and LWD may exist, the terms MWD and LWD often are used interchangeably. For the purposes of this disclosure, the term LWD will be used with the understanding that this term encompasses both the collection of formation parameters and the collection of information relating to the movement and position of the drilling assembly.

The terms “couple” or “couples” and “connect” or “connects” as used herein are intended to mean either an indirect or a direct connection. Thus, if a first device couples or connects to a second device, that coupling or connection may be through a direct coupling or connection or through an indirect mechanical or electrical coupling or connection via other devices and coupling or connections. Similarly, the term “communicatively coupled” as used herein is intended to mean either a direct or an indirect communication connection. Such connection may be a wired or wireless connection such as, for example, Ethernet or LAN. Such wired and wireless connections are well known to those of ordinary skill in the art and will therefore not be discussed in detail herein. Thus, if a first device communicatively couples to a second device, that connection may be through a direct connection, or through an indirect communication connection via other devices and connections. The indefinite articles “a” or “an,” as used herein, are defined herein to mean one or more than one of the elements that it introduces. The term “fluid,” as used herein, is not limiting and can be used interchangeably to describe a gas, a liquid, a solid, or some combination of a gas, a liquid, and/or a solid.

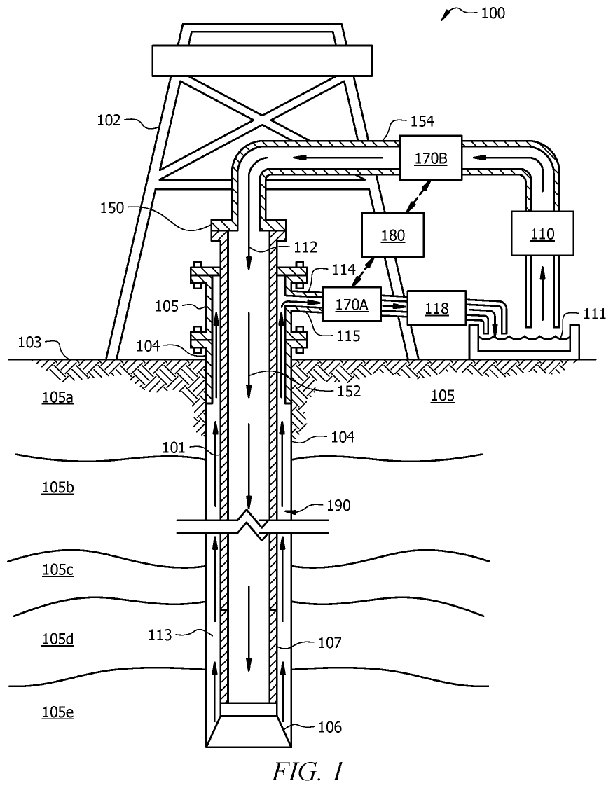

FIG. 1 is a diagram illustrating an example drilling system 100, according to aspects of the present disclosure. The drilling system 100 comprises a drilling assembly 190 that is suspended from a drilling rig 102 at the surface 103 and disposed in a borehole 104 within a formation 105. The formation 105 may be comprised of at least one rock strata. In the embodiment shown, the formation 105 is comprised of rock strata 105a-e, each of which may be made of different rock types with different characteristics. At least some of the strata 105a-e may be porous and contain trapped fluids and gases.

The drilling assembly 190 may comprise a tubular drill string 101 and a drill bit 106 may be coupled to a distal end of the drill string 101. The drill bit 190 may be rotated either by a top drive or kelley mechanism 150 at the surface 103 that rotates the entire drilling assembly 190, or by a downhole motor (not shown) to extend the borehole 104. In the embodiment shown, the drilling assembly 190 further comprises a bottom-hole assembly (BHA) 107 through which the drill bit 104 is indirectly coupled to the drill string 101. The BHA 107 may include a variety of MWD/LWD tools, drill collars, steering systems, downhole motors, etc., depending on the drilling application.

The drill string 101 extends downwardly through a surface tubular 108 into the borehole 104. The surface tubular 108 may be coupled to a wellhead 109. The wellhead 109 may include a portion that extends into the borehole 104. In embodiments, the wellhead 109 may be secured within the borehole 104 using cement, and may work with the surface tubular 108 and other surface equipment, such as a blowout preventer (BOP) (not shown), to prevent excess pressures from the formation 105 and borehole 104 from being released at the surface 103.

During drilling operations, a pump 110 located at the surface 103 may pump drilling fluid from a fluid reservoir 111 through the top drive 150, into the inner bore 152 of the drill string 101. The pump 110 may be in fluid communication with the inner bore 152 through at least one fluid conduit or pipe 154 coupled between the pump 110 and the top drive 150. As indicated by arrows 112, the drilling fluid may flow through the interior bore 152 of drill string 101, through the drill bit 106 and into a borehole annulus 113. The borehole annulus 113 is created by the rotation of the drill string 101 and attached drill bit 106 in borehole 104 and is defined as the space between the interior/inner wall or diameter of borehole 104 and the exterior/outer surface or diameter of the drill string 101. The annular space may extend out of the borehole 104, through the wellhead 109 and into the surface tubular 108.

The drilling fluid can be any drilling fluid. For example, in embodiments, the drilling fluid can be or comprise a water based drilling fluid, a non-water based drilling fluid (e.g., diesel oil, mineral oil, synthetic fluid), an emulsion, or a combination thereof. In embodiments, the drilling fluid comprises a water based drilling fluid as the term is understood to a person of ordinary skill in the wellbore drilling arts. In embodiments, the drilling fluid is a water based drilling fluid wherein water (or saltwater) is the majority liquid phase (e.g., greater than 50 weight percent (wt %)) as well as the wetting (external) phase (e.g., the phase wetting solid surfaces such as drill cuttings). In embodiments, the sampled drilling fluid comprises a water based drilling fluid comprising water, clay, a polymer emulsifier, and one or more viscosifiers.

Fluid pumped into the borehole annulus 113 through the drill string 101 flows upwardly through the borehole annulus 113. Surface tubular 108 is in fluid communication with the borehole annulus 113 and the drilling fluid may exit the borehole annulus 113 into the annular space of the surface tubular 108. The surface tubular 108 may have an outlet port 114 coupled to a fluid conduit or pipe 115. The fluid conduit 115 may also be referred to as a fluid return, where drilling fluid pumped downhole through the drill string 101 returns to the surface 103. Specifically, drilling fluid flowing through the borehole annulus 113 may enter the surface tubular 108 and exit through the outlet 114 to the fluid conduit 115. The fluid conduit 115 may provide fluid communication between the borehole annulus 113 and at least one fluid treatment mechanism or “bulk solids removal” 118, which may include screens that filter out bulk particulates from the fluid before passing the fluid to the surface reservoir 111.

According to aspects of the present disclosure, the drilling system comprises at least one fluid analyzer 170A/170B (also referred to herein as an “apparatus”) that is in fluid communication with the drilling fluid as it enters the internal bore 152 of the drill string 101 and/or as it exits the borehole 104 after flowing through the borehole annulus 113. In embodiments, the fluid analyzer may be in fluid communication with the drilling fluid by being either coupled to or in fluid communication with the interior of one or both of fluid conduits 115 and 154. For example, gas can be monitored going into and coming out of the wellbore with mirrored setups, in embodiments. In alternative or additional embodiments, the fluid analyzer 170A/170B may be in fluid communication with the drilling fluid being either coupled to or in fluid communication with a fluid tank, fluid line, possum belly, gumbo box, return line, suction line, stand pipe, or other point at the well head. Desirably, the apparatus is positioned adjacent the wellbore or borehole 104 (e.g., adjacent fluid conduit 115) and upstream of any bulk solids removal 118.

The fluid analyzer 170A/170B may comprise a stand-alone machine or mechanism or may comprise integrated functionality of a larger analysis/extraction mechanism. FIG. 1 shows a first fluid analyzer or apparatus 170A in fluid communication with the fluid conduit 115 between the surface tubular 108 and the fluid treatment mechanism 118. In embodiments, the fluid analyzer 170A may comprise at least one probe that is inserted into the fluid conduit 115 to provide fluid communication with the drilling fluid exiting the borehole annulus 113. In other embodiments, the fluid conduit 115 may comprise multiple segments that are connected directly to the fluid analyzer 170A, which may partially act as a portion of the fluid conduit 115 and take fluid samples/measurements as needed.

FIG. 1 further shows a second fluid analyzer or apparatus 170B in fluid communication with the fluid conduit 154 between pump 110 and the top drive 150. In embodiments, the fluid analyzer 170B may comprise at least one probe that is inserted into the fluid conduit 154 to provide fluid communication with the drilling fluid entering the internal bore 154 of the drill string 101. In other embodiments, the fluid conduit 154 may comprise multiple segments that are connected directly to the fluid analyzer 170B, which may partially act as a portion of the fluid conduit 154 and take fluid samples/measurements as needed. Although two fluid analyzers are shown in FIG. 1, only one fluid analyzer may be used in other embodiments, positioned similarly to either one of the fluid analyzers 170A and 170B.

At least some of the strata 105a-e may contain trapped fluids that are held under pressure. As the borehole 104 penetrates new strata, some of these fluids may be released into the borehole 104. The released fluids may become suspended or dissolved in the drilling fluid as it exits the drill bit 106 and travels through the borehole annulus 113. Each released fluid may be characterized by its chemical composition, and certain formation strata may be identified by the fluids it contains. As will be described below, the fluid analyzers 170A and 170B may take periodic or continuous samples of the drilling fluid, for example, by pumping, gravity drain or diversion of flow, or other means. The fluid analyzers 170A and 170B can generate corresponding measurements of the samples that may be used to determine the chemical composition of the drilling fluid. This chemical composition can be utilized to determine the types of fluid that are suspended within the drilling fluid, which can then be used to determine a formation characteristic about the formation 105.

In embodiments, the fluid analyzers 170A and 170B can be communicably coupled to an information handling system 180 positioned, for example, at the surface 103. The information handling system 180 can receive an output (including a GC output, as detailed further hereinbelow) from the fluid analyzers 170A and 170B and/or control the operation of the fluid analyzers 170A and 170B, including how often the fluid analyzers 170A and 170B take measurements (e.g., how often a drilling fluid sample is introduced into a GC system of the fluid analyzer 170A/170B, rather than being sent to bypass). In embodiments, the information handling system 180 can be dedicated to the fluid analyzer(s) 170A/170B. In embodiments, the information handling system 180 can receive measurements from a variety of devices in the drilling system 100 and/or control the operation of other devices.

The output of the fluid analyzer(s) 170A/170B can correspond to measurements taken by the fluid analyzer(s) 170A/170B of the drilling fluid or of samples of the drilling fluid. In embodiments, the information handling system 180 can determine a chemical composition of the drilling fluid using the fluid analyzer(s) 170A/170B, and in particular from the outputs (including a GC output, as detailed further hereinbelow) from fluid analyzer(s) 170A/170B. The chemical composition of the drilling fluid may comprise one or more types of chemicals found in the drilling fluid and the relative concentrations thereof. The information handling system 180 can determine the chemical composition, for example, by receiving an output from the fluid analyzer(s) 170A/170B, and comparing the output to a data set (e.g., a first data set) corresponding to known chemical compositions. The information handling system 180 can further determine the types of fluid suspended within the drilling fluid based on the determined chemical composition.

In embodiments, the information handling system 180 can determine a formation characteristic using the determined chemical composition. An example determined chemical composition for a drilling fluid may be 15% chemical/compound A, 20% chemical/compound B, 60% chemical/compound C, and 5% other chemicals/compounds. Example downhole characteristics include, but are not limited to, the type of rock in the formation 105, the presences of hydrocarbons in the formation 105, the types of hydrocarbons in the formation 105, the production potential for a strata 105a-e of the formation 105, the movement of fluid within a strata 105a-e, or a combination thereof. In embodiments, the information handling system 180 can determine the formation characteristic using the determined chemical composition characteristics by comparing the determined chemical composition to a data set (e.g., a second data set) that includes chemical compositions of known subterranean formations. For example, the determined chemical composition may correspond to a drilling fluid with suspended fluid from a shale layer in the formation 105.

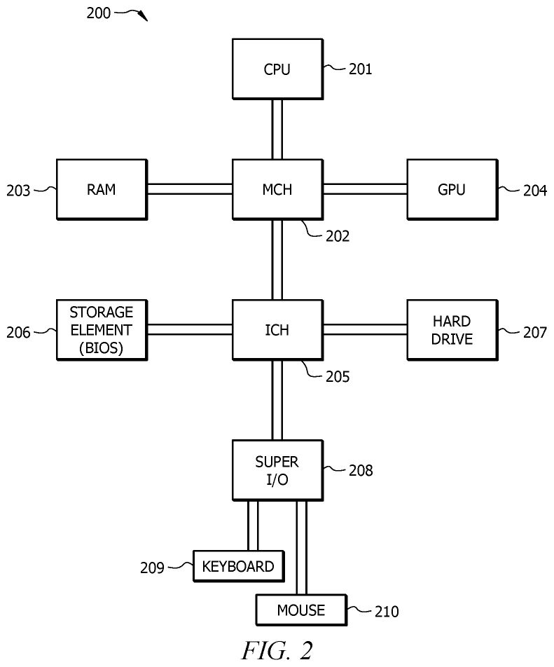

FIG. 2 is a block diagram showing an example information handling system 200, according to aspects of the present disclosure. A processor or CPU 201 of the information handling system 200 is communicatively coupled to a memory controller hub or north bridge 202. Memory controller hub 202 may include a memory controller for directing information to or from various system memory components within the information handling system, such as RAM 203, storage element 206, and hard drive 207. The memory controller hub 202 may be coupled to RAM 203 and a graphics processing unit 204. Memory controller hub 202 may also be coupled to an I/O controller hub or south bridge 205. I/O hub 205 is coupled to storage elements of the computer system, including a storage element 206, which may comprise a flash ROM that includes a basic input/output system (BIOS) of the computer system. I/O hub 205 is also coupled to the hard drive 207 of the computer system. I/O hub 205 may also be coupled to a Super I/O chip 208, which is itself coupled to several of the I/O ports of the computer system, including keyboard 209 and mouse 210. In embodiments, the Super I/O chip may also be connected to and receive input from a fluid analyzer, such as or similar to fluid analyzers 170A and 170B from FIG. 1.

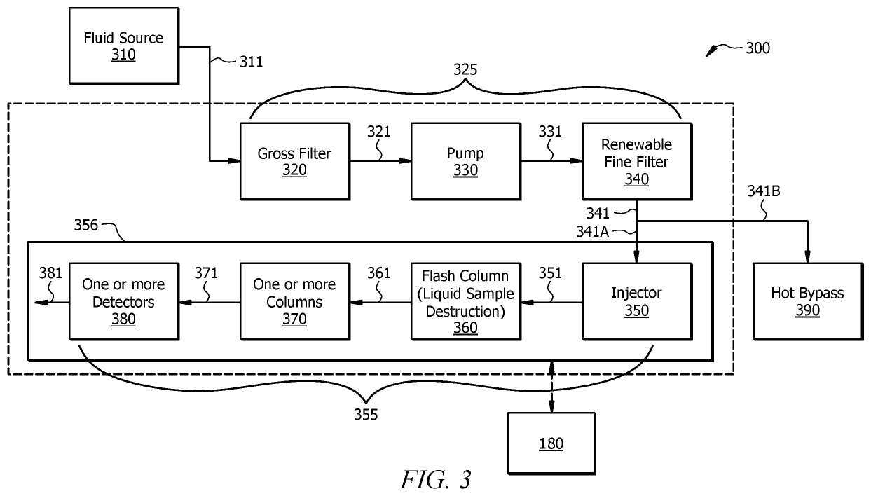

According to the present disclosure, a fluid analyzer or apparatus 170A/170B comprises a GC system. FIG. 3 is a block diagram illustrating an example fluid analyzer or apparatus 300 comprising a GC system 355 and suitable for use as analyzer 170A/170B, according to aspects of the present disclosure. The fluid analyzer or apparatus 300 may be in fluid communication with a fluid source 310. The fluid source 310 may comprise drilling fluid as it enters into or exits from the wellbore in a drilling system, for example the drilling fluid in fluid conduit 115 or fluid conduit 154, as described hereinabove with reference to FIG. 1.

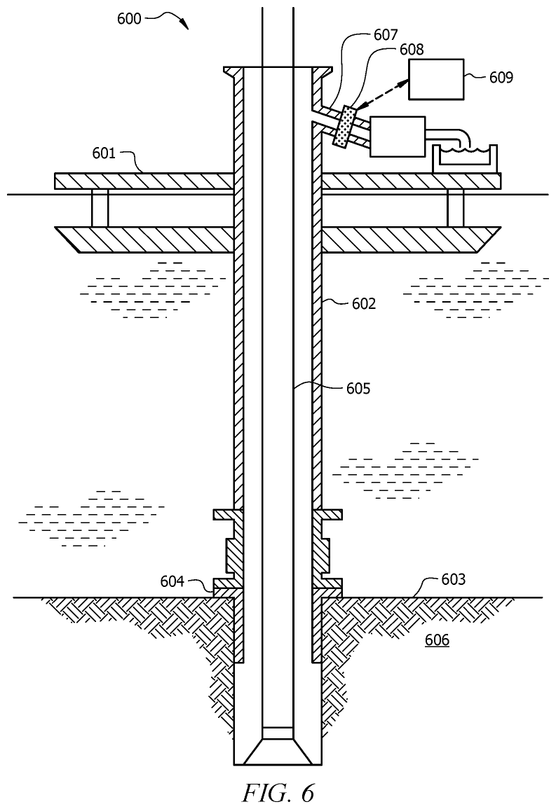

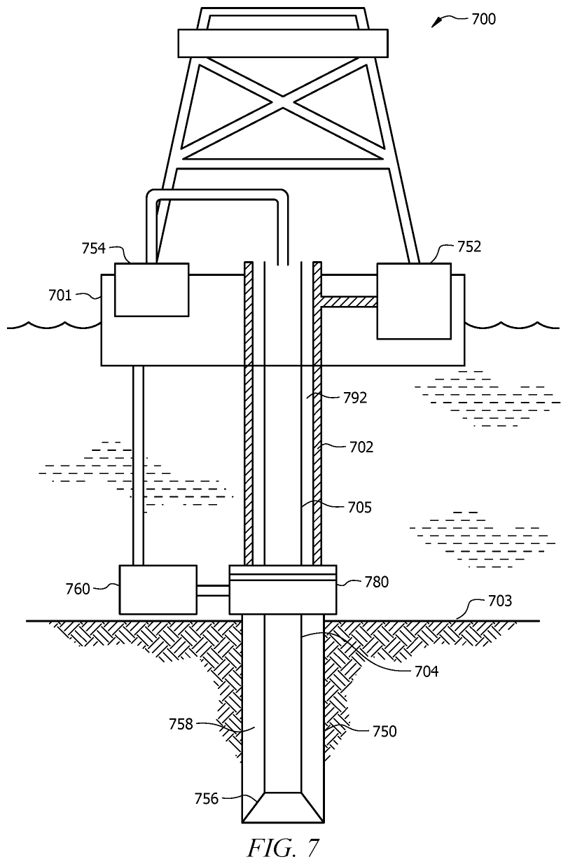

As depicted in FIG. 3, a system for analyzing a drilling fluid used in a drilling operation within a subterranean formation can comprise: a fluid analyzer 300 comprising a gas chromatograph (GC) system 355 in fluid communication with a drilling assembly (e.g., drilling assembly 100 of FIG. 1, 600 of FIG. 6, described hereinbelow, or 700 of FIG. 7, described hereinbelow) and configured to produce a GC output (indicated at 381); a bypass line 341B; and an information handling system 180, as described in FIG. 1, wherein the information handling system 180 is communicably coupled to the GC system 355, and comprises a processor 201 and a memory device (e.g., RAM 203, storage element 206, and/or hard drive 207) coupled to the processor 201, and the memory device contains a set of instructions that, when executed by the processor 201, cause the processor 201 to receive the output 381 of the GC system 355; and determine a chemical composition of the drilling fluid (drilling fluid flow indicated by arrows 112) using the output 381 of the GC system 355. A portion 311 of the drilling fluid is continuously extracted into analyzer 300 from a fluid conduit (e.g., fluid conduit 115, fluid conduit 154) coupled to the drilling assembly 100/600/700 and a drilling fluid sample 341 of the portion of the drilling fluid is introduced (e.g., via drilling fluid GC sample line 341A) to the GC system 335 or to a bypass line 341B to bypass 390 (e.g., disposal, collection with the remainder of the drilling fluid in fluid conduit 115/154 which was not extracted by analyzer 300, etc.).

The set of instructions that cause the processor 201 to determine the chemical composition of the drilling fluid using the output 381 of the GC system 355 can further cause the processor 201 to compare the output 381 of the GC system 355 to a data set corresponding to known chemical compositions.

The set of instructions, when executed by the processor 201, can further cause the processor 201 to determine a formation characteristic (i.e., a characteristic of the subterranean formation 105) using the determined chemical composition. The set of instructions that cause the processor 201 to determine the formation characteristic using the determined chemical composition can further cause the processor 201 to compare the determined chemical composition to a data set containing chemical compositions of known subterranean formations. The formation characteristic can comprise, without limitation, at least one of a type of rock in the formation 105, a presence of hydrocarbons in the formation 105, a type of hydrocarbons in the formation 105, a production potential for a strata 105a/e (e.g., stratum 105a, 105b, 105c, 105d, or 105e) of the formation 105, a movement of fluid within the strata 105a/e, or a combination thereof.

As depicted in FIG. 3, the GC system 355 can comprise an injector 350, a flash column 360, one or more gas chromatography columns 370 (referred to for simplicity as “a GC column 370”), and one or more detectors 380 (referred to for simplicity as “a detector 380”).

The injector 350 is operable to provide drilling fluid sample 351 from drilling fluid GC sample line 341A comprising a drilling fluid sample to be analyzed to flash column 360. The drilling fluid sample 351 can be automatically injected via injector 350 into flash column 360, as directed by information handling system 180, for example. Within flash column 360, the sample (which comprises liquid) is flashed to extract light hydrocarbons (e.g., methane, ethane, propane, butane, pentane, etc., C1-C5 or some combination thereof, as discussed further hereinbelow) therefrom, as a gaseous drilling fluid sample 361. By way of example, the gases extracted can include hydrocarbons such as, without limitation, methane, ethane, ethylene, propane, propylene, iso-butane, n-butane, neo-pentane, iso-pentane, n-pentane, hexane, heptane, octane, cyclo-hexane, methyl-cyclo-hexane, toluene, benzene, or others, and/or inorganic gases such as, without limitation, hydrogen, helium, carbon dioxide, nitrogen, or a combination thereof.

Flash column 360 can configured to (e.g., entirely) destruct the drilling fluid sample to provide a (e.g., an entirely) gaseous drilling fluid sample 361. Flash column 360 can be automatically replaced or regenerated, for example via high temperature flush with inert gas to clean the flash column 360. The flash column 360 can, for example, raise a temperature of the drilling fluid sample 351 from injector 350 to a temperature of greater than or equal to about 100, 200, or 250° C., or a temperature in a range of from about 100° C. to about 450° C., from about 200° C. to about 300° C., or from about 225° C. to about 275° C.

The gaseous drilling fluid sample 361 from flash column 360 can be introduced into the GC column 370. GC column 370 is configured to separate the chemical compounds in the gaseous drilling fluid sample 361 from flash column 360. The output 371 from the GC column 370 is detected by one or more detectors 380. The GC column 370 can be operable for the detection of hydrocarbons (e.g., light hydrocarbons), inorganics, isotopes (e.g., via gas chromatography/mass spectrometry (GC/MS)), etc.

The one or more detectors 380 can include a flame ionization detector (FID), a thermal conductivity detector (TCD), a pulse ionization detector (PID), another detector, or a combination thereof.

The GC output 381 from GC system 355 can be tagged to a depth in the wellbore and communicated to information handling system 180, as noted hereinabove. The information handling system (e.g., comprising a computer) can use the real time or depth-based data to determine a drilling efficiency and/or a fluid type in the formation 105.

Although the description of analyzer 300 focuses on the GC system thereof, it is to be understood that analyzer 300 can further comprise additional apparatus/columns that utilize other analytical techniques. For example, and without limitation, analyzer 300 can further comprise spectroscopy apparatus, such as an infrared (IR) spectroscopy, Fourier Transform Infrared Spectroscopy (FTIR), Raman spectroscopy, or a combination thereof.

The portion 311 of the drilling fluid can be continuously extracted from the fluid conduit via a sampling system 325, which can be separate from or a component of fluid analyzer 300. In embodiments, the portion 311 of the drilling fluid in the fluid conduit can be continuously extracted from the fluid conduit via a sample pump 330 of sampling system 325. A gross filter 320 of sampling system 325 can be positioned upstream of the sample pump 330 and a regenerative fine filter 340 of the sampling system 325 can be positioned downstream from the sample pump 330. The gross filter 320 and the regenerative fine filter 340 remove solids from the portion 311 of the drilling fluid continuously extracted from the fluid conduit. The regenerative fine filter 340 removes smaller particles from the portion 311 of the drilling fluid than the gross filter 320.

The sample pump 330 can be designed, chosen, and/or configured to continuously provide the portion 311 of the drilling fluid (e.g., to the gross filter 320) at a flow rate of less than or equal to about 3000, 2000, 1000, 900, 800, 700, 600, 500, 400, 300, 200, or 100 mL/min (e.g., from about 10 to about 500, from about 50 to about 300, from about 50 to about 200 mL/min, or from about 10, 20, 30, 40, 50, 60, 70, 80, 90, or 95 mL/min to about 1000, 900, 800, 700, 600, 500, 400, 300, 200, 100, or 50 mL/min).

Gross filter 320 and regenerative fine filter 340 are operable to remove solids from the portion 311 of the drilling fluid extracted from the fluid conduit. The gross filter 320, the regenerative fine filter, or both can be regenerative or automatically replaced, as needed. The gross filter 320 can be designed to remove relatively large particles (e.g., greater than or equal to about 1/16^{th} inch (1.6 mm), ⅛^{th} inch (3 mm), or ¼ inch (6.4 mm) from the portion 311 of the drilling fluid prior to introduction of the larger-particle reduced drilling fluid sample 321 to sample pump 330, while the regenerative fine filter 340 can remove smaller particles than the gross filter 320, in preparation for introduction into injector 350. For example, gross filter 320 can comprise filter holes in a range of from about 1/16^{th} inch (1.6 mm) to about ¼ inch (6.4 mm), for example about ⅛^{th} inch (3 mm). In embodiments, regenerative fine filter 340 removes particles greater than about 0.5, 0.6, 0.7, 0.8, 0.9, 1, 2, 3, 4, or 5 μm from the portion of the drilling fluid sample 331 passing therethrough to provide the drilling fluid sample 341 (or “prepared drilling fluid” 341) upstream of the GC system 355.

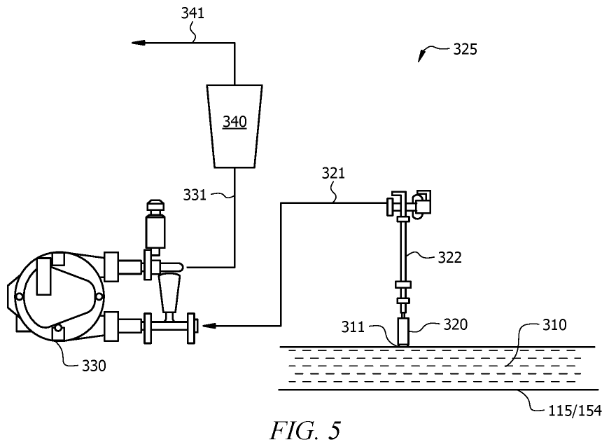

FIG. 5 is a schematic of an example sample preparation apparatus 325, according to aspects of the present disclosure. Upon return to the surface, a portion of the recovered drilling fluid can be extracted from the fluid conduit via sampling system 325 and the drilling fluid sample 341 bypassed around GC system 355 via bypass line 341B or the drilling fluid sample 341 can be fed to GC system 355 configured to gasify light hydrocarbons in the drilling fluid sample to provide a gaseous drilling fluid sample for GC analysis. Sampling system 325 can continuously extract portion 311 of the returned drilling fluid, for example via gross filter 320 and a suction tube assembly 322 in fluid communication with a source 310 of drilling fluid such as fluid conduit 115/154. The extracting/sampling via sampling system 325 can be continuous. A flow line 321 can be utilized to carry the portion 311 of the drilling fluid from sampling device 322 through sample pump 330, which sample pump 330 can further pump the portion 311 of the drilling fluid from which large particles have been removed in gross filter 320 into regenerative fine filter 340 via flow line 331. As noted above, the prepared drilling fluid sample 341 can be sent to bypass 390 or introduced into GC system 355.

The regenerative fine filter 240, the gross filter 320, or both can comprise multiple (e.g., two or more) filters in parallel, such that while one of the two filters is filtering the portion 311 of the drilling fluid to provide drilling fluid sample 341, the other of the two filters is being regenerated. Alternatively, for example, the regenerative fine filter 340 can comprise a roll of filtration material continuously drawn into a path of the drilling fluid, such that a fresh section of the roll of filtration material is continuously exposed to the continuously flowing portion 311 of the drilling fluid.

The apparatus 300 can be positioned adjacent to (e.g., within 1, 10, or 50 feet) a drilling system 100/600/700 (e.g., adjacent borehole 104 of the drilling system 100/600/700). As noted previously, the flow conduit from which the portion 311 of the drilling fluid is continuously extracted can be upstream of a bulk solids removal 118 of the drilling system, wherein the bulk solids removal 118 is configured to remove bulk solids from the drilling fluid.

In embodiments, an analyzer 300 for analyzing drilling fluid used in a drilling operation within a subterranean formation 105 comprises: gas chromatography (GC) system 355, as described hereinabove, and optionally a sampling system 325 and/or an information handling system 180 communicably coupled to the GC system 355, as described. The GC system 355 is in fluid communication with a fluid conduit (e.g., fluid conduit 115, fluid conduit 154) containing a drilling fluid source 310, and the fluid conduit is in fluid communication with a drilling assembly 100/600/700 at least partially disposed within the subterranean formation 105. The GC system 355 comprises injector 350, flash column 360, gas chromatography column 370, and detector 380, as described hereinabove. The flash column is configured to (e.g., entirely or at least partially partially) destruct a sample 351 of the drilling fluid to provide a gaseous sample 361, which gaseous sample 361 is introduced into the GC column 370. Light liquid hydrocarbons in sample 351 have been converted to gases in gaseous sample 361. As noted above, the information handling system 180 can be communicably coupled to the GC system 355, and can comprise a processor 201 and a memory device (e.g., (e.g., RAM 203, storage element 206, and/or hard drive 207) coupled to the processor 201, wherein the memory device contains a set of instructions that, when executed by the processor 201, cause the processor 201 to receive an output 381 of the GC system 355; determine a chemical composition of the drilling fluid within the fluid conduit using the output 381 of the GC system 355; and determine a formation 105 characteristic using the determined chemical composition. The information handling system 180 can further initiate sending of the prepared drilling fluid sample 341 either to the GC system 355 or bypass 390. For example, the prepared drilling fluid sample 341 can be automatically sent to GC system 355 periodically, at a certain depth of drilling, or randomly, or as directed by an operator, or a combination thereof.

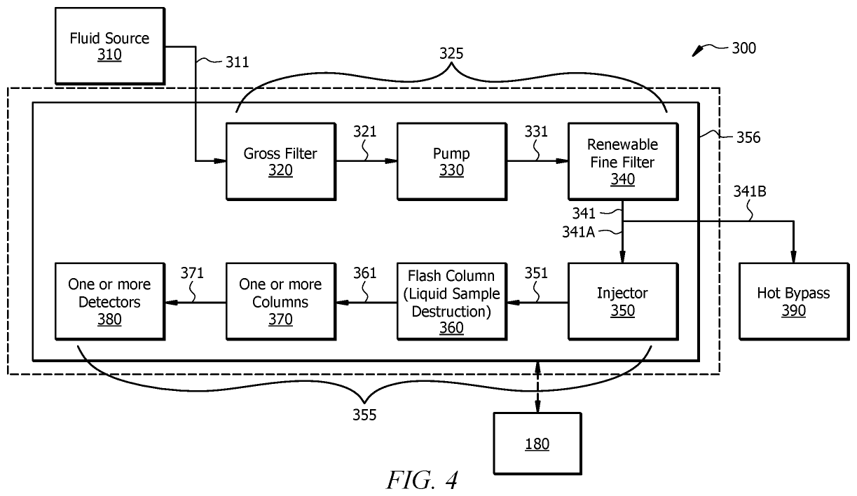

As depicted in FIG. 3, the injector 350, the flash column 360, the gas chromatography column 370, and the detector 380 of the GC system 355 can be positioned within a common housing 356 and/or otherwise provided as a portable unit (e.g., on a common frame for transport/positioning, etc.). As noted above, the fluid analyzer 300 can further comprise, fluidically upstream of the GC system 355, drilling fluid sampling apparatus 325 configured to provide a prepared sample 341A of the drilling fluid to the GC system 355. As described hereinabove, the drilling fluid sampling apparatus 325 can comprise a gross filter 320, a sample pump 330 downstream from the gross filter 320, and a regenerative fine filter 340 downstream from the sample pump 330, as described hereinabove. The gross filter 320, the pump 330, the regenerative fine filter 340, or a combination thereof can optionally also be positioned in the common housing 356. As depicted in FIG. 4, in embodiments, the injector 350, the flash column 360, the gas chromatography column 370, the detector 380 of the GC system 355 as well as the gross filter 320, the pump 330, and the regenerative fine filter 340, can be positioned within the common housing 356 and/or otherwise provided as a portable unit (e.g., on a common frame for transport/positioning, etc.).

The drilling fluid sampling apparatus 325 can be configured to continuously extract portion 311 of the drilling fluid from the flow conduit. The drilling fluid sampling apparatus 325 can be configured to continuously introduce extracted portion 311 of the drilling fluid from the flow conduit to the gross filter 320 and the regenerative fine filter 340 via the sample pump 330, to produce a prepared/filtered sample 341. The prepared/filtered sample 341 can be either introduced into the GC system 355 as the sample for GC analysis or can be sent to a bypass line 341B for removal from the system (e.g., to bypass 370).

As noted hereinabove, the sample pump 330 can be configured to provide the at least the portion 311 of the drilling fluid to the gross filter 320 at a flow rate of less than or equal to about 3000, 2000, 1000, 900, 800, 700, 600, 500, 400, 300, 200, or 100 mL/min (e.g., from about 10 to about 500, from about 50 to about 300, from about 50 to about 200 mL/min, or from about 10, 20, 30, 40, 50, 60, 70, 80, 90, or 95 mL/min to about 1000, 900, 800, 700, 600, 500, 400, 300, 200, 100, or 50 mL/min). This can be substantially lower than a flow rate needed by other conventional apparatus utilized to analyze drilling fluid composition.

By extracting the portion 311 of the drilling fluid from fluid conduit 115 proximate a location at which the drilling fluid exits the borehole 104, upstream of any bulk solids separation apparatus 118 configured to remove one or more components (e.g., bulk solids) from the drilling fluid, delays in fluid analysis (and discrepancies in predicted drilling fluid sample location or strata 105 a/e from which the (e.g., light) hydrocarbons in the drilling fluid were obtained) typically seen in conventional drilling fluid analysis can be minimized.

In embodiments, the information handling system 180 can be communicably coupled to at least one of the injector, the flash column 360, the one or more GC columns 370, the one or more detectors 380, or a combination thereof. The information handling system 180 can direct commands to and/or receive measurements from at least one of the injector, the flash column 360, the one or more GC columns 370, or the one or more detectors 380. The pump 330 may be coupled to and/or in fluid communication with at least a portion of the GC system 355. In embodiments, the pump 320, which can handle viscous fluids with entrained solids, can comprise at least one of a positive displacement pump, a peristaltic pump. or a scroll pump. Other pumps may be utilized, as will be appreciated by one of ordinary skill in the art in view of this disclosure.

In embodiments, the output 381 of the one or more detectors 380 may comprise the output of the GC system 355. In other embodiments, the output 381 of the one or more detectors 380 can be processed before it leaves the GC system 355. For example, an information handling system, similar to information handling system 180 and dedicated to the GC system 355, may be coupled to the one or more detectors 380 and may convert the output 381 of the one or more detectors in some way prior to transmittal to information handling system 180.

Once the chemical composition of the drilling fluid is determined as described above, the fluids suspended within the drilling fluid (e.g., returned to the surface 103 along with the drilling fluid introduced downhole) may be determined by excluding those chemicals known to have been in the drilling fluid before the drilling fluid was introduced downhole. Additionally, once the types of fluid suspended within the drilling fluid are known, those fluids and corresponding chemical compositions may be correlated to a data set corresponding to known chemical compositions of subterranean formations, allowing for formation characteristics about the subterranean formation to be determined.

Although the fluid analyzer 170A/170B/300 comprising GC has been described herein in the context of a conventional drilling assembly positioned at the surface, the fluid analyzer may similarly be used with different drilling assemblies (e.g., wirelines, slickline, etc.) in different locations. For example, FIG. 6 is a diagram of an offshore drilling system 600, according to aspects of the present disclosure. As can be seen, portions of the drilling system 600 may be positioned on a floating platform 601. A tubular 602 may extend from the platform 601 to the sea bed 603, where the well head 604 is located. A drill string 605 may be positioned within the tubular 602, and may be rotated to penetrate the formation 606. Drilling fluid may be circulated downhole within the drill string 605 and return to the surface in an annulus between the drill string 605 and the tubular 602. A proximal portion of the tubular 602 may comprise a fluid conduit 607 coupled thereto. The fluid conduit 607 may function as a fluid return, and a fluid analyzer 608, according to aspects of the present disclosure (e.g., a fluid analyzer 170A/170B/300), may be coupled to the fluid conduit 607 and/or in fluid communication with a drilling fluid within the fluid conduit 607. Likewise, the fluid analyzer 608 may be communicably coupled to an information handling system 609 (which can be an information handling system 180, as described herein) positioned on the platform 601.

By way of further example, FIG. 7 is a diagram of a dual gradient offshore drilling system 700, according to aspects of the present disclosure. As can be seen, portions of the drilling system 700 may be positioned on a floating boat or platform 701. A riser 702 may extend from the platform 701 to the sea bed 703, where the well head 704 is located. A drill string 705 may be positioned within the riser 702 and a borehole 750 within the formation 706. The drill string 705 may pass through a sealed barrier 780 between the riser 702 and the borehole 705. The annulus 792 surrounding the drill string 705 within the riser 702 may be filled with sea water, and a first pump 752 located at the surface may circulate sea water within the riser 702. A second pump 754 positioned at the platform 701 may pump drilling fluid through the drill string 705. Once the drilling fluid exits the drill bit 756 into annulus 758, a third pump 760, located underwater, may pump the drilling fluid to the platform 701. A fluid analyzer 170A/170B/300 of this disclosure may be incorporated at various locations within the system 700, including, without limitation, in fluid communication with fluid conduits between pumps 754 and 760, or in fluid communication with fluid conduits between the pumps 754 and 760 and the drill string 705.

According to aspects of the present disclosure, an example method for analyzing drilling fluid used in a drilling operation within a subterranean formation may comprise placing a GC system 355 and/or a fluid analyzer 170A/170B/300 of this disclosure in fluid communication with a drilling fluid. The drilling fluid may be flowing through a fluid conduit coupled to a drilling assembly 100/600/700. A chemical composition of the drilling fluid may be determined using the GC system 355. A formation characteristic may be determined using the determined chemical composition.

In embodiments, a method for analyzing a drilling fluid used in a drilling operation within a subterranean formation 105 according to this disclosure comprises: flowing the drilling fluid through a fluid conduit 115/154 coupled to a drilling assembly 100/600/700; continuously flowing a portion 311 of the drilling fluid comprising a drilling fluid sample 341 of the drilling fluid from the fluid conduit, wherein the drilling fluid sample comprises a liquid; sending the drilling fluid sample 341 to a bypass 341B, or determining a chemical composition of the drilling fluid using gas chromatography (GC) of the drilling fluid sample 341. Determining the chemical composition of the drilling fluid using GC comprises: introducing the drilling fluid sample 341 via line 341A into GC system 355, and destructing (e.g., flashing to convert light liquid hydrocarbons to gas) the drilling fluid sample to provide a gaseous drilling fluid sample 361, wherein destructing the drilling fluid sample comprises converting at least a portion of the components of the drilling fluid sample to a gas; and introducing the gaseous drilling fluid sample 361 to one or more GC column 370 to determine the chemical composition of the drilling fluid.

As discussed hereinabove, determining the chemical composition of the drilling fluid using the GC can comprise receiving an output 381 of a GC system 355 at an information handling system 180 coupled to the GC system 355; and comparing the output 381 of the GC system 355 to a data set corresponding to known chemical compositions.

As noted herein, the method can further comprise determining a characteristic of a subterranean formation 105 using the determined chemical composition. Determining the formation characteristic using the determined chemical composition can comprise comparing the determined chemical composition to a data set corresponding to known chemical compositions of subterranean formations. The formation 105 characteristic can comprise a type of rock in the formation 105, a presence of hydrocarbons in the formation 105, a type of hydrocarbons in the formation 105, a production potential for a strata 105a-e of the formation, a movement of fluid within the strata 105a-e, or a combination thereof.

Continuously extracting the drilling fluid sample 341 from the fluid conduit 115/154 can further comprise continuously pumping, via a sample pump 330, a portion 311 of the drilling fluid from the drilling fluid conduit 115/154 through a gross filter 320 upstream of the sample pump 330 and a regenerative fine filter 340 downstream from the sampling pump 330 to provide a continuous flow of the drilling fluid sample 341, which can either be sent to GC system 355 for analysis in GC system 355 or sent to bypass 390.

As noted hereinabove, the regenerative fine filter 340 can comprise multiple (e.g., two or more) filters in parallel, such that one of the two filters is filtering the portion 311 of the drilling fluid to provide the drilling fluid sample 341, while the other of the two filters is being regenerated, or wherein the regenerative fine filter 340 comprises a roll of filtration material continuously drawn into a path of the portion 311 of the drilling fluid, such that a fresh section of the roll of filtration material is continuously exposed to the continuously flowing drilling fluid. In embodiments, the regenerative fine filter 340 removes particles greater than about 1 μm from the portion 311 of the drilling fluid upstream of an injector 350 of the GC system 355.

The sample pump 330 can continuously provide the portion 311 of the drilling fluid to the gross filter 320 at a flow rate of less than or equal to about 3000, 2000, 1000, 900, 800, 700, 600, 500, 400, 300, 200, or 100 mL/min (e.g., from about 10 to about 500, from about 50 to about 300, from about 50 to about 200 mL/min, or from about 10, 20, 30, 40, 50, 60, 70, 80, 90, or 95 mL/min to about 1000, 900, 800, 700, 600, 500, 400, 300, 200, 100, or 50 mL/min).

As detailed hereinabove with reference to FIG. 1, the fluid conduit containing the fluid source 310 from which the drilling fluid sample 341 is obtained can comprise an annulus 113 in a wellbore 104 or a flow conduit (e.g., flow conduit 115, flow conduit 154) fluidly connected therewith. In applications, the fluid conduit contains the drilling fluid exiting a wellbore 104 of the drilling system 100/600/700 and upstream of any bulk solids separation apparatus 118 configured to remove one or more components (e.g., bulk solids) from the drilling fluid.

Destructing the drilling fluid sample 341 (injected by injector 350 as drilling fluid sample 351 into flash chamber 361) to provide the gaseous drilling fluid sample 361 can comprise introducing the drilling fluid sample 341A/351 to a flash column 360, and increasing the drilling fluid sample to a temperature above a boiling point of every component of the drilling fluid sample, such that substantially all of the light liquid hydrocarbons therein or substantially an entirety of the drilling fluid sample is converted to a gas as the gaseous drilling fluid sample 361.

In embodiments, determining the chemical composition of the drilling fluid using the GC may comprise receiving an output of the GC system 355 at an information handling system 180 coupled to the GC system 355; and comparing the output of the GC system to a first data set corresponding to known chemical compositions. Similarly, determining the formation characteristic using the determined chemical composition may comprise comparing the determined chemical composition to a second data set corresponding to known chemical compositions of subterranean formations. The formation 105 characteristic may comprise at least one of a type of rock in the formation 105, the presence of hydrocarbons in the formation 105, the type(s) of hydrocarbons in the formation 105, the production potential for a strata 105 a-e of the formation 105, and/or the movement of fluid within a strata 105a-e.

In embodiments, a method of analyzing drilling fluid at a wellsite according to this disclosure comprises: continuously withdrawing a drilling fluid sample stream from a source of the drilling fluid; filtering the drilling fluid sample stream to provide a filtered drilling fluid sample stream; routing the filtered drilling fluid sample stream to a by-pass valve; when the by-pass valve is open, routing the filtered drilling fluid sample stream to a by-pass line; when the by-pass valve is closed, routing the filtered drilling fluid sample stream to a flash chamber, wherein all or a portion of at least one liquid component of the drilling fluid is vaporized; and subjecting the vaporized component of the drilling fluid to gas chromatography.

In embodiments, a method of analyzing a drilling fluid at a wellsite according to this disclosure comprises: continuously withdrawing a drilling fluid sample stream from a source of the drilling fluid at the wellsite; periodically flashing the drilling fluid sample stream to vaporize all or a portion of at least one liquid component of the drilling fluid; and analyzing the vaporized component of the drilling fluid via gas chromatography.

In embodiments, a method of analyzing a drilling fluid at a wellsite according to this disclosure comprises: continuously withdrawing a drilling fluid sample stream from a source of the drilling fluid at the wellsite; gasifying all or a portion of at least one liquid component of the drilling fluid to provide one or more gaseous components of the drilling fluid; and analyzing the one or more gaseous components via gas chromatography.

The method can further comprise filtering the drilling fluid sample stream through filter media prior to the flashing. The filtering can remove substantially all (e.g., greater than 90, 91, 92, 93, 94, 95, 96, 97, 98, 99, 99.9, or 99.99 wt. %) of solids from the drilling fluid sample stream. The method can further comprise regenerating the filter media.

The drilling fluid can comprise a plurality of liquid components that are vaporized during the flashing or gasifying and wherein an identity of the vaporized/gaseous liquid components, an amount of the vaporized/gaseous liquid components, or both is determined via the gas chromatography. In embodiments, the identity, the amount, or both are determined via a flame ionization detector (FID), a thermal conductivity detector (TCD), a photo ionization detector (PID), another detector, or a combination thereof

Analyzer 170A/170B/300 of this disclosure can be utilized to extract drilling fluid, for example, from the circulated, recovered drilling fluid (e.g., water based mud) returned to the surface 103, and subject the extracted drilling fluid to a variety of analytical techniques (e.g., columns and detectors) including gas chromatography and optionally one or more of mass spectrometry, isotopic analysis, total hydrocarbon analysis, or a combination thereof. The data obtained from the detectors can be subjected to further geochemical analysis for formation fluid characterization (e.g., identification, quantification) and further reservoir insight (e.g., hydrocarbon extraction efficiency, porosity, permeability, compartmentalization, etc.). The process of extracting and analyzing hydrocarbons from drilling fluid that has been circulated from the surface, downhole, and returned back to the surface and recovered from the wellbore can also be referred to as surface data logging (SDL), surface mud logging, surface data logging-while-drilling (LWD), surface mud logging-while-drilling (LWD), and the like.

The system and method of this disclosure can provide for determination of formation properties (e.g., surface data logging). The determination of formational hydrocarbons (i.e., hydrocarbons in a subterranean formation) is conventionally limited in the gas phase due to extraction. The fluid analyzer, system and method described herein can overcome current extraction bias, and enable liquid phase detection (e.g., detection/compositional determination of liquid hydrocarbons in drilling fluid) in a gas phase.

The herein disclosed system and method utilize GC continuously coupled to a liquid flow of drilling fluid from the wellbore. The GC system includes a flash column to fully extract hydrocarbons (e.g., light hydrocarbons, such as C1-C5 hydrocarbons) from a drilling fluid sample 351 for introduction into the GC column(s). The GC system 355 can be positioned adjacent the wellbore or borehole 104, upstream of any bulk solids removal 118, in embodiments.

The herein disclosed fluid analyzer, system and method enable the chemical analysis to be performed without human intervention (e.g., it can be automated, as described herein). In embodiments, no extraction efficiency correction (EEC) for correction of gas extraction bias is utilized in the chemical analysis described herein.

### Additional Disclosure

The following are non-limiting, specific embodiments in accordance with the present disclosure:

In a first embodiment, a method for analyzing a drilling fluid used in a drilling operation within a subterranean formation comprises: flowing the drilling fluid through a fluid conduit coupled to a drilling assembly; continuously flowing a drilling fluid sample of the drilling fluid from the fluid conduit, wherein the drilling fluid sample comprises a liquid; sending the drilling fluid sample to a bypass, or determining a chemical composition of the drilling fluid using gas chromatography (GC) of the drilling fluid sample, wherein determining the chemical composition of the drilling fluid using GC comprises: destructing the drilling fluid sample to provide a gaseous drilling fluid sample, wherein destructing the drilling fluid sample comprises converting one or more components (e.g., light hydrocarbons) of the drilling fluid sample to a gas; and introducing the gaseous drilling fluid sample to a GC system comprising a GC column to determine the chemical composition of the drilling fluid.

A second embodiment can include the method of the first embodiment further comprising determining a formation characteristic using the determined chemical composition.

A third embodiment can include the method of the second embodiment, wherein determining the formation characteristic using the determined chemical composition comprises comparing the determined chemical composition to a data set corresponding to known chemical compositions of subterranean formations.

A fourth embodiment can include the method of the second or third embodiment, wherein the formation characteristic comprises a type of rock in the formation, a presence of hydrocarbons in the formation, a type of hydrocarbons in the formation, a production potential for a strata of the formation, a movement of fluid within the strata, or a combination thereof.

A fifth embodiment can include the method of any one of the first to fourth embodiments, wherein determining the chemical composition of the drilling fluid using the GC comprises receiving an output of a GC system at an information handling system coupled to the GC system; and comparing the output of the GC system to a data set corresponding to known chemical compositions.

A sixth embodiment can include the method of any one of the first to fifth embodiments, wherein continuously flowing the drilling fluid sample from the fluid conduit further comprises continuously pumping, via a sample pump, a portion of the drilling fluid from the drilling fluid conduit through a gross filter upstream of the sample pump and a regenerative fine filter downstream from the sampling pump to provide a continuous flow of the drilling fluid sample, wherein the gross filter and the regenerative fine filter remove solids from the portion of the drilling fluid, and wherein the regenerative fine filter removes smaller particles from the portion of the drilling fluid sample than the gross filter.

A seventh embodiment can include the method of the sixth embodiment, wherein the regenerative fine filter comprises multiple (e.g., two or more) filters in parallel, such that one of the two filters is filtering the drilling fluid sample while the other of the two filters is being regenerated, or wherein the regenerative fine filter comprises a roll of filtration material continuously drawn into a path of the drilling fluid sample, such that a fresh section of the roll of filtration material is continuously exposed to the continuously flowing drilling fluid sample.

An eighth embodiment can include the method of the sixth or seventh embodiment, wherein the regenerative fine filter removes particles greater than about 1 μm from the drilling fluid sample upstream of an injector of the GC system.

A ninth embodiment can include the method of any one if the sixth to eighth embodiments, wherein the sample pump provides the portion of the drilling fluid to the gross filter at a flow rate of less than or equal to about 3000, 2000, 1000, 900, 800, 700, 600, 500, 400, 300, 200, or 100 mL/min (e.g., from about 10 to about 500, from about 50 to about 300, from about 50 to about 200 mL/min, or from about 10, 20, 30, 40, 50, 60, 70, 80, 90, or 95 mL/min to about 1000, 900, 800, 700, 600, 500, 400, 300, 200, 100, or 50 mL/min).

A tenth embodiment can include the method of any one of the first to ninth embodiments, wherein the fluid conduit comprises an annulus in a wellbore or a flow conduit fluidly connected therewith.

An eleventh embodiment can include the method of the tenth embodiment, wherein the fluid conduit contains the drilling fluid exiting a wellbore of the drilling operation and upstream of any bulk solids separation apparatus configured to remove one or more components (e.g., bulk solids) from the drilling fluid.

A twelfth embodiment can include the method of any one of the first to eleventh embodiments, wherein destructing the drilling fluid sample to provide the gaseous drilling fluid sample comprises introducing the drilling fluid sample to a flash column, and increasing the drilling fluid sample to a temperature above a boiling point of (e.g., every component of) the drilling fluid sample, such that (e.g., substantially an entirety of) the drilling fluid sample is converted to a gas as the gaseous drilling fluid sample.

In a thirteenth embodiment, a system for analyzing a drilling fluid used in a drilling operation within a subterranean formation comprises: a gas chromatograph (GC) system in fluid communication with a drilling assembly and configured to produce a GC output; a bypass line; and an information handling system communicably coupled to the GC system, wherein the information handling system comprises a processor and a memory device coupled to the processor, and the memory device contains a set of instructions that, when executed by the processor, cause the processor to receive the output of the GC system; and determine a chemical composition of the drilling fluid using the output of the GC system, wherein a portion of the drilling fluid is continuously extracted from a fluid conduit coupled to the drilling assembly and a drilling fluid sample of the portion of the drilling fluid is introduced to the GC system or the bypass line.

A fourteenth embodiment can include the system of the thirteenth embodiment, wherein the set of instructions, when executed by the processor, further cause the processor to determine a formation characteristic using the determined chemical composition.

A fifteenth embodiment can include the system of the fourteenth embodiment, wherein the set of instructions that cause the processor to determine the formation characteristic using the determined chemical composition further cause the processor to compare the determined chemical composition to a data set containing chemical compositions of known subterranean formations.

A sixteenth embodiment can include the system of the fourteenth or fifteenth embodiment, wherein the formation characteristic comprises at least one of a type of rock in the formation, a presence of hydrocarbons in the formation, a type of hydrocarbons in the formation, a production potential for a strata of the formation, a movement of fluid within the strata, or a combination thereof.

A seventeenth embodiment can include the system of any one of the thirteenth to sixteenth embodiments, wherein the set of instructions that cause the processor to determine the chemical composition of the drilling fluid using the output of the GC system further cause the processor to compare the output of the GC system to a data set corresponding to known chemical compositions.

An eighteenth embodiment can include the system of any one of the thirteenth to seventeenth embodiments, wherein the GC system comprises an injector, a flash column, a gas chromatography column, and a detector, wherein the flash column is configured to (e.g., entirely) destruct the drilling fluid sample to provide a (e.g., an entirely) gaseous drilling fluid sample, and wherein the gaseous drilling fluid sample is introduced into the GC column.

A nineteenth embodiment can include the system of any one of the thirteenth to eighteenth embodiments, wherein the portion of the drilling fluid is continuously extracted from the fluid conduit via a sample pump, and wherein a gross filter is positioned upstream of the sample pump and a regenerative fine filter is positioned downstream from the sample pump, wherein the gross filter and the regenerative fine filter remove solids from the portion of the drilling fluid, and wherein the regenerative fine filter removes smaller particles from the portion of the drilling fluid sample than the gross filter.

A twentieth embodiment can include the system of the nineteenth embodiment, wherein the regenerative fine filter removes particles greater than about 1 μm from the portion of the drilling fluid sample passing therethrough to provide the drilling fluid sample upstream of the GC system.

A twenty first embodiment can include the system of the nineteenth or twentieth embodiment, wherein the sample pump is configured to provide the portion of the drilling fluid to the gross filter at a flow rate of less than or equal to about 3000, 2000, 1000, 900, 800, 700, 600, 500, 400, 300, 200, or 100 mL/min (e.g., from about 10 to about 500, from about 50 to about 300, from about 50 to about 200 mL/min, or from about 10, 20, 30, 40, 50, 60, 70, 80, 90, or 95 mL/min to about 1000, 900, 800, 700, 600, 500, 400, 300, 200, 100, or 50 mL/min).

A twenty second embodiment can include the system of any one of the nineteenth embodiment to twenty first embodiments, wherein the apparatus is positioned adjacent to (e.g., within 5, 10, 20, 30, 40, 50, or 100 feet) a wellbore of the drilling system, and wherein the flow conduit from which the portion of the drilling fluid is continuously extracted is upstream of a bulk solids removal of the drilling operation, wherein the bulk solids removal is configured to remove bulk solids from the drilling fluid.

In a twenty third embodiment, an analyzer for analyzing a drilling fluid used in a drilling operation within a subterranean formation comprises: a gas chromatography (GC) system, wherein the GC system is in fluid communication with a fluid conduit, wherein the fluid conduit is in fluid communication with a drilling assembly at least partially disposed within the subterranean formation, wherein the GC system comprises an injector, a flash column, a gas chromatography column, and a detector, wherein the flash column is configured to (e.g., entirely) destruct a sample of the drilling fluid to provide a gaseous sample, and wherein the gaseous sample is introduced into the GC column.

A twenty fourth embodiment can include the analyzer of the twenty third embodiment, wherein the injector, the flash column, the gas chromatography column, and the detector of the GC system are positioned within a common housing.

A twenty fifth embodiment can include the analyzer of the twenty fourth embodiment further comprising, upstream of the GC system, drilling fluid sampling apparatus configured to provide the sample of the drilling fluid to the GC system, wherein the drilling fluid sampling apparatus comprises a gross filter, a sample pump downstream from the gross filter, and a regenerative fine filter downstream from the sample pump, wherein the gross filter and the regenerative fine filter remove solids from a portion of the drilling fluid continuously extracted from the fluid conduit, and wherein the regenerative fine filter removes smaller particles from the portion of the drilling fluid than the gross filter.

A twenty sixth embodiment can include the analyzer of the twenty fifth embodiment, wherein the injector, the flash column, the gas chromatography column, and the detector of the GC system are positioned within a common housing, and wherein the gross filter, the pump, the regenerative fine filter, or a combination thereof is or is not positioned in the common housing.

A twenty seventh embodiment can include the analyzer of the twenty fifth or twenty sixth embodiment, wherein the drilling fluid sampling apparatus is configured to continuously introduce a portion of the drilling fluid from the flow conduit to the gross filter and the regenerative fine filter via the sample pump, to produce a filtered sample, and wherein the filtered sample is either introduced into the GC as the sample or sent to a bypass line for removal from the system.

A twenty eighth embodiment can include the analyzer of the twenty seventh embodiment, wherein the sample pump is configured to provide the at least the portion of the drilling fluid to the gross filter at a flow rate of less than or equal to about 3000, 2000, 1000, 900, 800, 700, 600, 500, 400, 300, 200, or 100 mL/min (e.g., from about 10 to about 500, from about 50 to about 300, from about 50 to about 200 mL/min, or from about 10, 20, 30, 40, 50, 60, 70, 80, 90, or 95 mL/min to about 1000, 900, 800, 700, 600, 500, 400, 300, 200, 100, or 50 mL/min).

A twenty ninth embodiment can include the analyzer of any one of the twenty third to twenty eighth embodiments, wherein the fluid conduit contains the drilling fluid flowing out of a wellbore of the drilling operation, upstream of any bulk solids separation apparatus configured to remove one or more components (e.g., bulk solids) from the drilling fluid.

In a thirtieth embodiment, a method of analyzing drilling fluid at a wellsite comprises: continuously withdrawing a drilling fluid sample stream from a source of the drilling fluid; filtering the drilling fluid sample stream to provide a filtered drilling fluid sample stream; routing the filtered drilling fluid sample stream to a by-pass valve; when the by-pass valve is open, routing the filtered drilling fluid sample stream to a by-pass line; when the by-pass valve is closed, routing the filtered drilling fluid sample stream to a flash chamber, wherein all or a portion of at least one liquid component of the drilling fluid is vaporized; and subjecting the vaporized component of the drilling fluid to gas chromatography.

In a thirty first embodiment, a method of analyzing a drilling fluid at a wellsite comprises: continuously withdrawing a drilling fluid sample stream from a source of the drilling fluid at the wellsite; periodically flashing the drilling fluid sample stream to vaporize all or a portion of at least one liquid component of the drilling fluid; and analyzing the vaporized component of the drilling fluid via gas chromatography.

In a thirty second embodiment, a method of analyzing a drilling fluid at a wellsite comprises: continuously withdrawing a drilling fluid sample stream from a source of the drilling fluid at the wellsite; gasifying all or a portion of at least one liquid component of the drilling fluid to provide one or more gaseous components of the drilling fluid; and analyzing the one or more gaseous components via gas chromatography.

A thirty third embodiment can include the method of the thirty first or thirty second embodiment further comprising filtering the drilling fluid sample stream through filter media prior to the flashing.

A thirty fourth embodiment can include the method of any one of the thirtieth to thirty second embodiments, wherein the filtering removes substantially all (e.g., greater than 90, 91, 92, 93, 94, 95, 96, 97, 98, 99, 99.9, or 99.99 wt. %) of solids from the drilling fluid sample stream.

A thirty fifth embodiment can include the method of the thirty third or thirty fourth embodiment further comprising regenerating the filter media.

A thirty sixth embodiment can include the method of any one of the thirtieth to thirty fifth embodiments, wherein the drilling fluid comprises a plurality of liquid components that are vaporized during the flashing or gasifying and wherein an identity of the vaporized/gaseous liquid components, an amount of the vaporized/gaseous liquid components, or both is determined via the gas chromatography.

A thirty seventh embodiment can include the method of the thirty sixth embodiment, wherein the identity, the amount, or both are determined via a flame ionization detector (FID), a thermal conductivity detector (TCD), a photo ionization detector (PID), another detector, or a combination thereof.

While embodiments have been shown and described, modifications thereof can be made by one skilled in the art without departing from the spirit and teachings of this disclosure. The embodiments described herein are exemplary only, and are not intended to be limiting. Many variations and modifications of the embodiments disclosed herein are possible and are within the scope of this disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R1, and an upper limit, Ru, is disclosed, any number falling within the range is specifically disclosed. In particular, the following numbers within the range are specifically disclosed: R=R1+k*(Ru−R1), wherein k is a variable ranging from 1 percent to 100 percent with a 1 percent increment, i.e., k is 1 percent, 2 percent, 3 percent, 4 percent, 5 percent, 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of broader terms such as comprises, includes, having, etc. should be understood to provide support for narrower terms such as consisting of, consisting essentially of, comprised substantially of, etc. When a feature is described as “optional,” both embodiments with this feature and embodiments without this feature are disclosed. Similarly, the present disclosure contemplates embodiments where this “optional” feature is required and embodiments where this feature is specifically excluded.

Accordingly, the scope of protection is not limited by the description set out above but is only limited by the claims which follow, that scope including all equivalents of the subject matter of the claims. Each and every claim is incorporated into the specification as embodiments of the present disclosure. Thus, the claims are a further description and are an addition to the embodiments of the present disclosure. The discussion of a reference herein is not an admission that it is prior art, especially any reference that can have a publication date after the priority date of this application. The disclosures of all patents, patent applications, and publications cited herein are hereby incorporated by reference, to the extent that they provide exemplary, procedural, or other details supplementary to those set forth herein.

What is claimed is:

1. A method for analyzing a drilling fluid used in a drilling operation within a subterranean formation, the method comprising:
flowing the drilling fluid through a fluid conduit coupled to a drilling assembly;
continuously flowing a drilling fluid sample of the drilling fluid from the fluid conduit, wherein the drilling fluid sample comprises a liquid;
sending the drilling fluid sample to a bypass, or determining a chemical composition of the drilling fluid using gas chromatography (GC) of the drilling fluid sample, wherein determining the chemical composition of the drilling fluid using GC comprises:
destructing the drilling fluid sample to provide a gaseous drilling fluid sample, wherein destructing the drilling fluid sample comprises converting one or more components of the drilling fluid sample to a gas; and
introducing the gaseous drilling fluid sample to a GC system comprising a GC column to determine the chemical composition of the drilling fluid.

2. The method of claim 1 further comprising determining a formation characteristic using the determined chemical composition.

3. The method of claim 2, wherein determining the formation characteristic using the determined chemical composition comprises comparing the determined chemical composition to a data set corresponding to known chemical compositions of subterranean formations.

4. The method of claim 1, wherein determining the chemical composition of the drilling fluid using the GC comprises receiving an output of a GC system at an information handling system coupled to the GC system; and comparing the output of the GC system to a data set corresponding to known chemical compositions.

5. The method of claim 1, wherein continuously flowing the drilling fluid sample from the fluid conduit further comprises continuously pumping, via a sample pump, a portion of the drilling fluid from the drilling fluid conduit through a gross filter upstream of the sample pump and a regenerative fine filter downstream from the sample pump to provide a continuous flow of the drilling fluid sample, wherein the gross filter and the regenerative fine filter remove solids from the portion of the drilling fluid, and wherein the regenerative fine filter removes smaller particles from the portion of the drilling fluid sample than the gross filter.

6. The method of claim 5, wherein the regenerative fine filter comprises multiple filters in parallel, such that one of the two filters is filtering the drilling fluid sample while the other of the two filters is being regenerated, or wherein the regenerative fine filter comprises a roll of filtration material continuously drawn into a path of the drilling fluid sample, such that a fresh section of the roll of filtration material is continuously exposed to the continuously flowing drilling fluid sample.

7. The method of claim 5, wherein the sample pump provides the portion of the drilling fluid to the gross filter at a flow rate of less than or equal to about 3000 mL/min.

8. The method of claim 1, wherein the fluid conduit comprises an annulus in a wellbore or a flow conduit fluidly connected therewith.

9. The method of claim 8, wherein the fluid conduit contains the drilling fluid exiting a wellbore of the drilling operation and upstream of any bulk solids separation apparatus configured to remove one or more components from the drilling fluid.

10. The method of claim 1, wherein destructing the drilling fluid sample to provide the gaseous drilling fluid sample comprises introducing the drilling fluid sample to a flash column, and increasing the drilling fluid sample to a temperature above a boiling point of the drilling fluid sample, such that the drilling fluid sample is converted to a gas as the gaseous drilling fluid sample.

11. A system for analyzing a drilling fluid used in a drilling operation within a subterranean formation, the system comprising:
a gas chromatograph (GC) system in fluid communication with a drilling assembly and configured to produce a GC output;
a bypass line; and
an information handling system communicably coupled to the GC system, wherein the information handling system comprises a processor and a memory device coupled to the processor, and the memory device contains a set of instructions that, when executed by the processor, cause the processor to receive the output of the GC system; and determine a chemical composition of the drilling fluid using the output of the GC system,
wherein a portion of the drilling fluid is continuously extracted from a fluid conduit coupled to the drilling assembly and a drilling fluid sample of the portion of the drilling fluid is introduced to the GC system or the bypass line.

12. The system of claim 11, wherein the set of instructions that cause the processor to determine the chemical composition of the drilling fluid using the output of the GC system further cause the processor to compare the output of the GC system to a data set corresponding to known chemical compositions.

13. The system of claim 11, wherein the GC system comprises an injector, a flash column, a gas chromatography column, and a detector, wherein the flash column is configured to destruct the drilling fluid sample to provide a gaseous drilling fluid sample, and wherein the gaseous drilling fluid sample is introduced into the GC column.

14. The system of claim 11, wherein the portion of the drilling fluid is continuously extracted from the fluid conduit via a sample pump, and wherein a gross filter is positioned upstream of the sample pump and a regenerative fine filter is positioned downstream from the sample pump, wherein the gross filter and the regenerative fine filter remove solids from the portion of the drilling fluid, and wherein the regenerative fine filter removes smaller particles from the portion of the drilling fluid sample than the gross filter.

15. The system of claim 14, wherein the apparatus is positioned adjacent to a wellbore of the drilling system, and wherein the flow conduit from which the portion of the drilling fluid is continuously extracted is upstream of a bulk solids removal of the drilling operation, wherein the bulk solids removal is configured to remove bulk solids from the drilling fluid.

16. An analyzer for analyzing a drilling fluid used in a drilling operation within a subterranean formation, the analyzer comprising:
a gas chromatography (GC) system, wherein the GC system is in fluid communication with a fluid conduit, wherein the fluid conduit is in fluid communication with a drilling assembly at least partially disposed within the subterranean formation, wherein the GC system comprises an injector, a flash column, a gas chromatography column, and a detector, wherein the flash column is configured to destruct a sample of the drilling fluid to provide a gaseous sample, and wherein the gaseous sample is introduced into the GC column.

17. The analyzer of claim 16, wherein the injector, the flash column, the gas chromatography column, and the detector of the GC system are positioned within a common housing.

18. The analyzer of claim 17, further comprising, upstream of the GC system, drilling fluid sampling apparatus configured to provide the sample of the drilling fluid to the GC system, wherein the drilling fluid sampling apparatus comprises a gross filter, a sample pump downstream from the gross filter, and a regenerative fine filter downstream from the sample pump, wherein the gross filter and the regenerative fine filter remove solids from a portion of the drilling fluid continuously extracted from the fluid conduit, and wherein the regenerative fine filter removes smaller particles from the portion of the drilling fluid than the gross filter.

19. The analyzer of claim 18, wherein the injector, the flash column, the gas chromatography column, and the detector of the GC system are positioned within a common housing, and wherein the gross filter, the pump, the regenerative fine filter, or a combination thereof is or is not positioned in the common housing.

20. The analyzer of claim 18, wherein the drilling fluid sampling apparatus is configured to continuously introduce a portion of the drilling fluid from the flow conduit to the gross filter and the regenerative fine filter via the sample pump, to produce a filtered sample, and wherein the filtered sample is either introduced into the GC as the sample or sent to a bypass line for removal from the system.
